(19) United States
(12) Patent Application Publication
(10) Pub. No.: US 2019/0099114 A1
(43) Pub. Date: Apr. 4, 2019

(54) FALL SENSING AND MEDICAL ALERT SYSTEMS

(71) Applicant: Sensogram Technologies, Inc., Plano, TX (US)

(72) Inventors: Vahram Mouradian, Plano, TX (US); Qing Sun, Plano, TX (US); Tim Cogan, Plano, TX (US)

(21) Appl. No.: 16/142,535

(22) Filed: Sep. 26, 2018

Related U.S. Application Data
(60) Provisional application No. 62/565,431, filed on Sep. 29, 2017.

(51) Int. Cl. A61B 5/11; A61B 5/00; A61B 5/0205; A61B 5/024; A61B 5/053

(52) U.S. Cl. CPC A61B 5/1117; A61B 5/0002; A61B 5/0205; A61B 5/02438; A61B 5/01; A61B 5/1112; A61B 5/681; A61B 5/7465; A61B 5/746; A61B 5/053

(57) ABSTRACT

A fall detection device having a wearable housing, a processor, a sensor system operatively connected to the processor and configured to detect a physiological condition of the wearer's body, a motion sensor, a wireless communication module processor, and a memory. The memory stores computer readable instructions that cause the processor to: monitor the motion sensor to detect a decrease in acceleration in the gravitational direction below a first threshold; start a timer upon detecting the decrease in acceleration; monitor the motion sensor to detect a fall-indicative acceleration above a second level prior to the timer reaching a first predetermined time; monitor the motion sensor to detect a recovery-indicative acceleration above a third level prior to the timer reaching a second predetermined time; and activate the wireless communication module to initiate an emergency alert if the fall-indicative acceleration is detected and the recovery-indicative acceleration is not detected.

The application claims the benefit of U.S. Provisional Application No. 62/565,431, filed on Sep. 29, 2017, which is incorporated herein by reference in its entirety.

### TECHNICAL FIELD

The invention generally relates to wearable biosensors for detecting vital signs of the person wearing the device.

### BACKGROUND OF THE INVENTION

Everyday personal falling events represent a significant danger, particularly for persons with physical conditions that render them more susceptible to injury or less able to deal with the consequences of falling. Emergency rooms regularly address persons with fall-related injuries, and many of these injuries are fatal. Certain user-wearable devices, such as the neck-worn pendant device sold under the trade name LIFE ALERT by Life Alert Emergency Response, Inc. of Encino, Calif., provide a wireless transmitter that a user manually operates to call a local base station. The base station then contacts emergency services to request assistance. Such devices do not function if the wearer is not able to activate the device due to injury or unconsciousness, or if the wearer lacks the presence of mind to do so (e.g., due to dementia, shock, or forgetting that the device is being worn).

Biosensor systems are used to detect vital signs in the human body. These systems have been provided in a number of forms, from simple manually-operated stethoscopes and sphygmomanometers, to complex electronic monitoring systems. Early electronic biosensors were connected to the wearer and physically wired to monitoring equipment, making it difficult or impossible for the patient to move around during monitoring. More recently, electronic biosensors have been integrated into portable wearable devices that allow user mobility. For example, a typical wrist-mounted biosensor device has a housing that may be secured to a wearer by a band, much like a conventional wristwatch. An optical sensor system faces the user's wrist and includes an optical emitter that directs light into the wearer's wrist region, and an optical receiver that senses light reflected from the wrist region. A display, such as an interactive touchscreen or the like, is provided for observing data gathered by the optical sensor system. A suitable control and analysis system is provided in the device for controlling the optical sensor system to collect vital sign data, analyzing the vital sign data, and generating the desired output. The device also may include wireless communication systems, a battery, a charging port, a wired communication port, and so on. Wearable biosensor devices may operate independently, or in conjunction with other devices. For example, a “watch” style biosensor may be connected via wireless communications to a smartphone or other computer to permit remote control, processing power, and data output capabilities.

A need still remains to provide alternative fall detection and emergency alert systems, and to advance the state of wearable biosensor device art.

### SUMMARY

In one exemplary aspect, there is provided a fall detecting device having a housing configured to be positioned adjacent a wearer's body, a processor, a sensor system operatively connected to the processor and configured to detect a physiological condition of the wearer's body, a motion sensor operatively connected to the processor, a wireless communication module operatively connected to the processor, and a memory operatively connected to the processor. The memory stores computer readable instructions that, when executed, cause the processor to: monitor the motion sensor to detect a decrease in acceleration in the gravitational direction below a first predetermined threshold; start a timer upon detecting the decrease in acceleration; monitor the motion sensor to determine whether the motion sensor signals a fall-indicative acceleration above a second predetermined level prior to the timer reaching a first predetermined time; monitor the motion sensor to determine whether the motion sensor signals a recovery-indicative acceleration above a third predetermined level prior to the timer reaching a second predetermined time; and activate the wireless communication module to initiate an emergency alert if the fall-indicative acceleration is detected and the recovery-indicative acceleration is not detected.

The recovery-indicative acceleration may be an average value of accelerations over time.

The computer readable instructions may further cause the processor to activate a speaker to prompt the wearer to confirm or dismiss an initiation of an emergency alert.

The sensor system may include a photoplethysmographic sensor. The computer readable instructions may further cause the processor to: operate the photoplethysmographic sensor to detect blood flow information; determine a heart rate of the wearer based on the blood flow information; and display the heart rate on a user interface. The computer readable instructions may further cause the processor to: operate the photoplethysmographic sensor to detect a first set of blood flow information for a first detection time period following an initial activation of the fall detecting device; determine a first heart rate of the wearer during the first detection time period based on the first set of blood flow information; display the first heart rate on a user interface; operate the photoplethysmographic sensor to detect a second set of blood flow information for a second detection time period following the first detection time period; determine a second heart rate of the wearer during the second detection time period based on the second set of blood flow information; and display the second heart rate on the user interface. The second period of time may be longer than the first period of time.

The fall detecting device may have a user interface on the housing. The user interface may have a manually-operable emergency alert input operable to cause the processor to activate the wireless communication module to initiate an emergency alert. The user interface may be configured to provide visual information regarding the physiological condition of the wearer's body.

The fall detecting device may have a battery operatively connected to the processor and configured to power the fall detecting device.

The motion sensor may be a multi-axis accelerometer.

In another exemplary aspect, there is provided a fall detection method for a fall detecting device having at least one motion sensor. The method includes: monitoring the motion sensor to detect a decrease in acceleration in the gravitational direction below a first predetermined threshold; starting a timer upon detecting the decrease in acceleration; monitoring the motion sensor to determine whether the motion sensor signals a fall-indicative acceleration above a second predetermined level prior to the timer reaching a first predetermined time; monitoring the motion sensor to determine whether the motion sensor signals a recovery-indicative acceleration above a third predetermined level prior to the timer reaching a second predetermined time; and activating a wireless communication module to initiate an emergency alert if the fall-indicative acceleration is detected and the recovery-indicative acceleration is not detected.

The recovery-indicative acceleration may be an average value of accelerations over time.

The method may also include activating a speaker to request confirmation or dismissal of an initiation of an emergency alert.

### BRIEF DESCRIPTION OF THE DRAWINGS

Embodiments of the invention will now be described, strictly by way of example, with reference to the drawings identified below.

FIGS. 1A and 1B are front and rear isometric views, respectively, of an example of a wearable biosensor device incorporating one or more features as described herein.

FIG. 2 is a schematic diagram of a processing system that may be used in examples of wearable biosensor devices.

FIG. 3 is a process flow chart of an exemplary embodiment of the invention.

### DESCRIPTION OF THE EMBODIMENTS

The following description provides examples of wearable biosensor devices that may be used for evaluating whether a wearer has fallen and alerting emergency services to assist a person who has fallen. Examples also may include various additional features, such as heart rate monitoring, respiration rate monitoring, and so on. The examples provided describe wrist-mounted wearable biosensor devices. It will be appreciated, however, that other examples of devices may be mounted at alternative locations on the wearer's body. Also, various other alterations and reconfigurations of the devices structure and functionality may be provided in other examples, without departing from the scope of the inventions described herein.

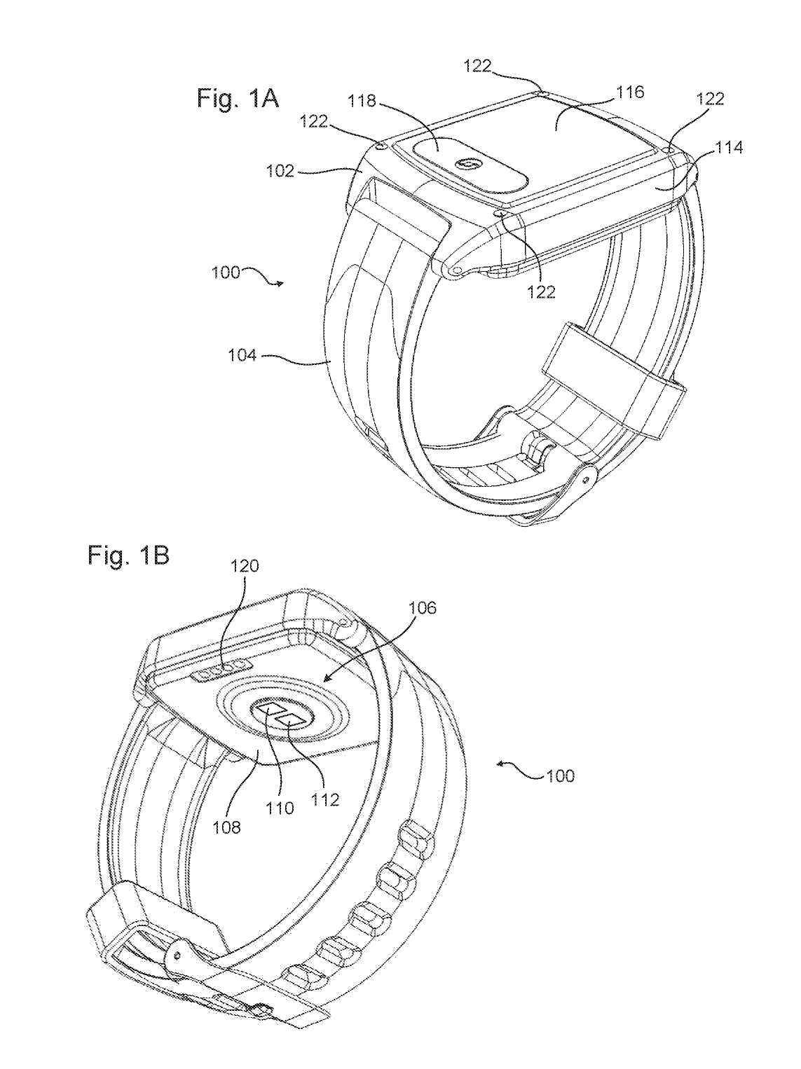

FIGS. 1A and 1B illustrate an example of a wearable biosensor device 100. The device 100 has a housing 102 configured to lie against the wearer's body, and a band 104 configured to hold the housing 102 against the wearer's body. The housing 102 is shaped and sized to fit on the desired target location for wearing the device 100, such as the wrist. The housing 102 provides a shell or platform to which the remaining parts are directly or indirectly attached. A plastic or metallic housing structure is expected to be suitable for most embodiments. For example, the housing 102 may be injection-molded plastic, cast magnesium, machined aluminum or steel, or the like. The housing 102 may include surface coatings or other features such as a water-resistant shell, a glass or transparent polycarbonate face, or the like. Other alternatives will be apparent to persons of ordinary skill in the art in view of the present disclosure.

The band 104 comprises a structure that wraps partially or entirely around the wearer's body to hold the housing 102 in place. In the example shown, the band 104 is configured to hold the housing 102 against the wearer's wrist. In other cases, the band 104 may be configured to hold the housing against the head, upper arm, hand, finger, leg, neck, waist, chest, or the like. The band 104 may comprise two flexible or rigid straps joinable by a clasp, a single elastic strap, two semi-rigid straps that form a “C” shape to partially encircle a body part, and so on. The band 104 may be movably or rigidly secured to the housing 102 by pivot pins, a cantilevered anchor, and so on. The band 104 also may be formed integrally with the housing 102.

When configured for use on the wrist, the housing 102 and band 104 preferably are configured with shapes and dimensions similar to a conventional wristwatch or smart watch. The housing 102 and band 104 also may comprise a conventional wristwatch or smart watch to which additional features such as discussed below are added to form an embodiment of the invention. In one example, the housing 102 may have a generally flat rectangular or rounded shape that extends in a plane with a maximum dimension in the plane of approximately two inches or less, and a thickness extending perpendicular to the plane of approximately one-half inch or less. The band 104 may be attached to edges of the housing 102 and configured to encircle a volume having a diameter of about two to three inches, or such a size as corresponds to the typical dimensions of a human wrist. The housing 102 optionally may be provided with conventional wristwatch features, such as a bezel, face and mechanical movement or digital clock for telling time.

The wearable biosensor device 100 also includes a sensor array 106. The sensor array 106 may include one or more sensors configured to collect vital sign information from the wearer, environmental information, and so on. One or more aspects of the sensor array 106 may be located at an inner surface 108 of the housing 102 (i.e., the surface facing the wearer's body during use). For example, the sensor array may comprise one or more optical emitters 110 located approximately centrally on inner surface 108 of the housing 102, and oriented to direct respective lights away from the inner surface 108 towards the wearer's body. The optical emitters 110 may direct the light at a 90° angle to the inner surface 108, or at an angle less than 90° thereto. Any desired number and pattern of optical emitters 110 may be used. Light emitting diodes (LEDs) are preferred for use as the optical emitters 110, but other light sources may be used in other embodiments.

The optical emitters 110 may emit light at one or more wavelengths. For example, a first group of one or more of the optical emitters 110 may emit light primarily at about 350-450 nanometers (green light), a second group of one of more of the optical emitters 110 may emit light primarily at about 605-750 nanometers (red light), and a third group of the one or more optical emitters 110 may emit light primarily at about 850-1020 nanometers (infrared light). The members of each group can be clustered together, or distributed among the other groups. The different groups can be operated simultaneously or separately, as desired. For example, the red light and infrared light groups can be alternatively activated to operate in a manner to cause oxyhemoglobin and deoxyhemoglobin in the blood to absorb the different light energies, and these energy levels can be compared to determine blood oxygen saturation, using techniques known in the art.

The sensor array 106 also includes one or more optical receivers 112 located in proximity to the optical emitter(s) 110. The optical receivers 112 are oriented to receive light reflected from the wearer's body and striking the inner surface 108, and may comprise any suitable device that is capable of determining the presence and/or intensity of such reflected light. Photodiodes, which produce a voltage or current proportional to the amount of impinging light energy, are preferred. The optical receivers 112 may be provided in any number of pattern.

The optical receivers 112 may be tuned to detect particular wavelengths of light. For example, a first group of optical receivers 112 may have a band-pass filter that only transmits light at a range of about 350-450 nanometers (green light), a second group of optical receivers 112 may have a band-pass filter that only transmits light at a range of about 605-750 nanometers (red light), and a third group of optical receivers 112 may have a band-pass filter that only transmits light at a range of about 850-1020 nanometers (infrared light). As another example, one or more of the optical receivers 112 may include a multi-band “knife-edge” filter that allows light at multiple discrete wavelengths to pass through (e.g., a filter that transmits light at one or more wavelengths within the range of 605-750 nanometers and one or more wavelengths within the range of 850-1020 nanometers). As still another example, the one or more optical receivers 112 may be unfiltered.

The signal detected by any one optical receiver 112 may be conditioned in various ways. For example, where the optical receiver 112 is unfiltered or includes a knife-edge filter that passes multiple different wavelengths, the signal from the optical receiver 112 can be demultiplexed to extract two different light signals corresponding to two different light sources being activated in an alternating sequence. Other alternatives will be apparent to persons of ordinary skill in the art in view of the present disclosure.

The sensor array 106 may be located at any suitable location on the inner surface 108. A location at the geometric middle of the inner surface 108 may provide improved shielding against ambient light, but this is not required. The sensor array 106 also may be located on a protuberance that extends away from the housing 102 relative to the adjacent portions of the inner surface 18, which can make it more likely that the sensor array 106 will rest firmly against the skin. Such a protuberance may act like a fulcrum that remains in contact with the skin as the housing 102 rocks through a range of motion on the wearer's body.

The housing 102 also has an outer surface 114 that does not face the wearer's body during use. The outer surface 114 may include an outer face that is generally parallel to the inner surface 108, sidewalls that extend from the outer face to the inner surface 108, and so on.

The device 100 may include one or more user interfaces, such as displays, user inputs, audio speakers, microphones, haptic feedback device (e.g., vibrators or tactile probes), and so on. For example, the outer surface 114 may have a display 116 configured to provide information to the wearer or a person assisting the wearer. An exemplary display 116 may comprise one or more indicating lights, such as light emitting diodes (LED), a two-dimensional LED screen, a two-dimensional liquid crystal display (LCD), and so on. An exemplary input may comprise a button 118, such as a capacitive button, a mechanical button, a momentary switch or the like. Multiple displays 116 and multiple buttons 118 also may be used. Functions of the displays 116 and inputs 118 are described in more detail below.

The device 100 also may include one or more charging ports, communication ports, or the like. For example, a mini-USB (universal serial bus) port may be provided on the inner surface 108 or outer surface 114 to selectively connect to a charging and/or communication cable. As another example, a dedicated charging port 120 may be provided on the inner surface 108 or the outer surface 114. The housing 102 also may include one or more charger mounts 122 that are configured to mate with a portable charging device, as discussed in more detail below.

It will be appreciated that the various components described as being part of the housing 102 may alternatively be moved to the band 104, or the band 104 and housing 102 may be integrated into a single continuous structure.

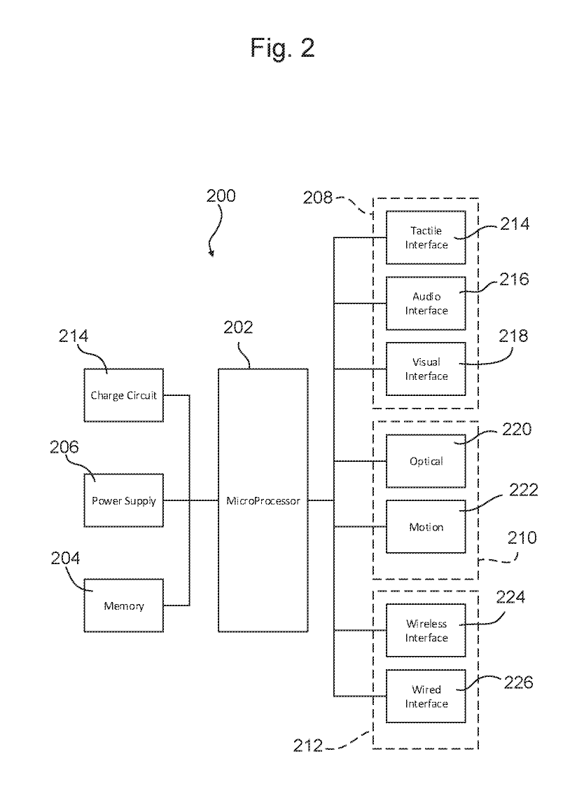

FIG. 2 is a schematic illustration of a operation system 200 that may be used with examples of wearable biosensor devices, such as the one shown in FIGS. 1A and 1B. In general terms, the operation system 200 is controlled by a computer processor 202 that is operatively connected to a memory 204, a power supply 206, a user interface system 208, a sensor system 210 (e.g., sensor array 106), and a communication system 212

The processor 202 is configured to execute computer-readable instructions stored on the memory 204. The memory 204 may be internal to the processor 202, or provided as a separate component. The processor 202 preferably is microprocessor having a low power consumption profile. An exemplary processor 202 is a microprocessor control unit based on the 32 bit ARM Cortex-M4 core, but any suitable processor may be used. The memory 204 may be internal to the processor 202 or external thereto. For example, the memory 204 may comprise any suitable digital memory storage system, such as a serial flash memory drive having a 256 Mb capacity. The particular details of the processor 202 and memory 204 need not be discussed in detail herein.

The power supply 206 may comprise a battery, capacitors, a wired power supply leading to an external power source, and so on. The power supply 206 preferably is a self-contained battery (e.g., lithium ion or nickel metal hydride) to provide high portability and a long life cycle. The battery preferably is rechargeable, but this is not strictly required. If a rechargeable battery or other rechargeable power supply 206 is used, the system 200 may include a dedicated charge circuit 214 comprising wiring, hardware, and electronic logic and control systems to control charging of the power supply 206, monitor the charge status of the power supply 206, and so on. The charge circuit 214 may be connected to one or more charging inputs that are configured to receive electric power. One charging input may be a wired charging port 120. Another charging input may be an inductive charging receiver, such as a secondary coil connected to a charging circuit that received electrical energy via resonant inductive coupling, as known in the art. Power supplies, charge circuits, and charging inputs are known in the art, and need not be discussed in detail herein.

The user interface system 208 may include any number and type of devices for receiving user input and providing information to the user. In one example, the device includes a tactile interface 214, an audio interface 216, and a visual interface 218.

The tactile interface 214 includes features that receive and transmit via touch. For example, as noted above, a biosensor device 100 may include one or more buttons 118 to receive user inputs. In one example, a single button 118 is provided on the outer surface 114 to make the device as simple as possible to use in an emergency situation. A single button 118 may be programmed to operate in different modes, depending on the pattern or duration of activation. For example, pressing the button 118 once briefly may turn on the display 116 for a certain period of time to observe visual information, and pressing it briefly again may turn off the display 116 to conserve power. Pressing the button 118 twice quickly may turn the device on or off. Pressing the button 118 for an extended period, such as three seconds or more, may initiate an emergency alert, as discussed below. Pressing the button 118 for an extended period after initiating an emergency alert may cancel the emergency alert. Alternatively, a single button 118 may have only the single purpose of being pressed to create an emergency alert (and optionally to cancel the emergency alert as well). Where a single button 118 is provided as an emergency alert button, the device 100 may be programmed to perform other functions (e.g., setting a clock or customizing the device to the wearer's preferences) via an interface with a smartphone, computer, or other remote terminal.

Other buttons also may be provided. Additional buttons preferably are located and configured to reduce the likelihood that a wearer will confuse those buttons with an emergency alert button. For example, a large emergency alert button may be provided on the main face of the outer surface 114, such as show in FIGS. 1A and 1B, and additional buttons (e.g., power, mode, programming, etc.) may be provided on the side faces of the outer surface 114 or on the inner surface 108. The tactile interface 214 also may include momentum- or orientation-detecting devices to receive input via physical manipulation of the device. For example, accelerometers may be used to detect deliberate movements for controlling device functions (e.g., shaking or turning the device over to step backwards in a menu system).

The tactile interface 214 also may include one or more tactile output devices, such as haptic feedback devices. For example, a motorized actuator with an offset weight may be provided inside the housing 102, and configured to operate to cause a vibration or movement shift to provide tactile information to the wearer by vibrating the housing 102. Such feedback may include, for example, vibrating continuously or in a repeating pattern to indicate when an emergency alert has been called, vibrating briefly to indicate when user input has been received, vibrating to indicate certain observed conditions have been met (e.g., pulse rate above a certain level), and so on.

The audio interface 216 may comprise any suitable speaker and/or microphone, as known in the art. For example, a speaker may be provided in the housing 102 and programmed to emit information in the form of audio output. Such output may include tones indicating that an emergency alert has been activated or deactivated, that an emergency authority is responding to the alert, and so on. The speaker also may be operable to transmit audio signals and to provide two way communication (along with a microphone) with emergency responders. For example, when an emergency alert is generated, the device 100 may be connected via wireless communications (e.g., a cellular telephone network) to an emergency services dispatcher (e.g., a local 9-1-1 call center), and telephonic communication may be made through a speaker and microphone within the device 100.

The visual interface 218 includes one or more devices to visually indicate information, such as LED screens or the like. The visual interface 218 also may include visual user input systems, such as gesture recognition devices or the like.

The sensor system 210 comprises one or more devices configured to evaluate the environment surrounding the wearable biosensor device 100. The sensor system 210 preferably includes an optical sensor 220 having one or more optical emitters 110 and optical receivers 112, such as described above. The optical sensor 220 also may include an ambient light detection circuit, optical filters, and other features.

The optical sensor 220 may be configured and programmed as a photoplethysmographic (PPG) device that detects volumetric flow of blood within the wearer's body at a location adjacent to the device 100. For example, the optical sensor 220 may have an optical emitter 110 that emits light towards the wearer's skin, and an optical receiver 112 that detects light reflected or absorbed by the blood flowing through the underlying tissue. One or more LEDs and associated detectors, such as those described above, may be used for this purpose, but other devices may be used in other examples. The volume of blood in the tissue adjacent to the device 100 changes during each heartbeat pressure pulse, and the optical receiver 112 generates a current or voltage output having a waveform generally corresponding to the change in flow volume. This is commonly referred to as PPG data. The PPG data from the optical sensor 220 may be used to provide heart rate information by evaluating the frequency of flow volume peaks. For example, the heart rate may be estimated by counting the number of flow maxima over time. Such techniques are generally known in the art, and need not be described herein in detail.

The accuracy of the heart rate estimation depends on the resolution of the waveform, which, in turn, depends on the sampling rate of the system. The sampling rate may be a function of the optical emitter 110 activation cycle, the optical receiver 112 activation cycle, the processor's 202 activation cycle, and so on. Higher sampling rates provide more detailed PPG data, and increase the ability to pinpoint the exact time of each volume flow peak. However, higher sampling rates also require more energy consumption to activate the optical emitter 110, poll the optical receiver 112, and perform the necessary data processing to extract each PPG data point.

It has also been found that the accuracy of the heart rate estimation varies with the length of the sample data set. Estimations based on short sample sets (e.g., five or ten heartbeats) can be significantly less accurate than estimations based on longer sample sets (e.g., fifty or sixty heartbeats). However, extremely long sample sets (e.g., one thousand heartbeats) also provide less accurate instantaneous measurements of heart rate because they can include sample data not reflective of the person's current condition. For example, extremely long data sets will react slowly to rapid changes in heart rate and may not accurately register brief, but significant, changes in heart rate. The selection of the sampling rate and data set length can affect the overall performance of the device 100 as a heart rate monitor, however, the balancing of such considerations is within the ordinary skill in the art and can be accomplished successfully without undue experimentation. Various known algorithms may be used to this end.

It is expected that users of a wearable biosensor device 100 may be more satisfied if the device 100 is able to begin providing heart rate measurements shortly after being worn or activated. To this end, a two-stage heart rate measuring algorithm may be used. When the device 100 is first activated, the processor 202 begins operating the optical emitter 110 and optical receiver 112 to collect PPG data. During the initial period of activation, the processor 202 analyzes the PPG data using a first heart rate algorithm based on a relatively short sample set, and begins outputting the results of this algorithm as soon as output information becomes available. This provides a relatively inaccurate heart rate estimation shortly after the device 100 begins operating. For example, the first algorithm may use a sample set comprising a 5-10 or 5-30 second rolling window of PPG data. Using this algorithm, the processor 202 can start providing heart rate estimations shortly after the initial window of data collection is complete. This is expected to provide a heart rate estimation that is accurate within about 10 beats per minute (bpm) of the actual heart rate for the period in question.

After a predetermined time has elapsed, the processor 202 changes to a second heart rate algorithm based on a relatively long sample set (i.e., longer than the first sample set discussed above) to provide a relatively accurate heart rate estimation. The second algorithm may, for example, use a sample set comprising a 10-60, 20-60 or 30-60 second rolling window of PPG data. The rolling window of data used by the second algorithm may begin at the time the device 100 is first activated, in which case the data used to perform the second algorithm may overlap the data used to perform the first algorithm. This minimizes the amount of time before the second algorithm takes over and start providing more accurate heart rate estimations. Using this algorithm the processor 202 may be able to start providing heart rate estimates about 35 to 40 seconds after the device is activated. The estimate provided by this second algorithm is expected to be accurate within about 1.0 bpm of the actual heart rate for the period in question. Once results from the second algorithm are available, the processor 202 may stop performing the first algorithm to conserve energy and processing power. The second algorithm may be used for the remaining duration of the device's 100 use, until it is removed from the wearer or rendered inactive.

The first and second algorithms may incorporate any algorithm that provides frequency data based on a measured waveform. In one example, the first and second algorithms evaluate frequency domain information from the PPG data using analytical processes such as Fast Fourier Transformations (FFT) to extract frequency domain peaks from the PPG data. Such peaks can then be filtered to identify pulse rate candidates (e.g., frequencies outside a certain range can be removed), and the pulse rate can then be selected as a remaining dominant peak. Other alternatives will be apparent to persons of ordinary skill in the art in view of the present disclosure.

The foregoing dual-stage heart rate algorithm process provides results that are likely to be relatively inaccurate during the initial operation period. However, the ability to provide heart rate estimations shortly after activating the device 100 is expected to be beneficial to satisfy the wearer's expected desire to measure his or her heart rate shortly after activating the device 100.

Data from the optical sensor 220 also may be used to determine respiration rate. Arterial blood pressure and peripheral venous pressure change during respiration. This variation manifests itself in PPG data as cyclical changes in flow rate that overlap the flow rate change caused by pulsatile variations. The respiration rate typically is significantly slower than the heart rate, which facilitates extracting the flow rate changes attributable to respiration using techniques such as Fourier transforms, autoregression, demodulation, and the like. Like heart rate estimations, such methods rely generally on evaluating a moving window of data. However, it has been proposed to perform real-time estimation of respiration rate using, for example, adaptive infinite impulse response filters.

In another example, the optical sensor 220 may be operated to detect blood oxygen level. In this case, the optical sensor 220 may have a first optical emitter 110 in the red light range, a second optical emitter 110 in the infrared light range, and a single optical receiver 112. The red and infrared optical detectors are operated asynchronously to irradiate the underlying body tissue, and the optical receiver 112 may be operated continuously to detect the intensity of red and infrared light reflected by the blood in the wearer's body. The signal from the optical receiver 112 is then demultiplexed according to the operation schedule of the two optical emitters 110, to determine which portions of the detected light intensity are attributable to reflections of the red light, and which portions of the detected light intensity are attributable to reflections of the infrared light. The ratio of red light reflection intensity to infrared light reflection intensity can then be used to determine the blood oxygen saturation level, because oxyhemoglobin and deoxyhemoglobin absorb different wavelengths of red and infrared light. Such techniques, commonly called pulse oximetry, are known in the art.

It will be appreciated that any suitable algorithm may be used to estimate pulse rate, respiration rate, oxygen level, and other vital signs, and various such algorithms are known in the art. Furthermore, examples of devices 100 may not be capable of or may not be programmed to estimating one or more of the foregoing vital signs.

The device 100 also may include features to discriminate when the device 100 is not being worn. Proximity sensors and temperature sensors may be used for this purpose, but such devices may be relatively susceptible to experiencing false positive readings. For example, when relying on a proximity sensor to determine whether the device 100 is being worn, a false positive may arise if the device is removed from the person and placed on a surface in contact with the proximity sensor, and the device 100 may continue to operate as if it still being worn.

Examples also may use the optical sensor 220 to determine whether the device 100 is being worn. For example, PPG data generated by the optical sensor 220 may be processed using a white noise detector to determine whether the data includes the expected characteristics of pulsatile volume flow variations. A white noise filter may comprise, for example, an algorithm that averages the amplitude value of the optical sensor 220 data, and identifies whether the data includes a regular periodic signal that passes back and forth through the average value within a particular range of frequency values (e.g., 10-15 times per second). Another white noise filter may comprise a Fourier transform filter that identifies whether the data from the optical sensor 220 includes significant peaks in certain ranges of the frequency domain suggestive of a human pulse. Other alternatives will be apparent to persons of ordinary skill in the art in view of the present disclosure.

As noted above, the accuracy of estimations based on PPG data received from the optical sensor 220 is, in part, a function of the overall minimum sampling rate. It is typical to operate a PPG device at relatively high sampling rates (e.g., 512 Hz) to provide the most accurate PPG data possible. However, it is expected that in the context of a fall detection device such high levels of accuracy may not be necessary. Thus, in some examples, the device 100 may be operated at a relatively low sampling rate (e.g., 100 Hz). This is expected to conserve battery power and provide longer service life between battery charging. In such an example, the device 100 also may be programmed to automatically switch to a higher sampling rate (e.g., 512 Hz) during specific events, such as when an emergency alert is generated. This can provide more detailed information on an as-needed basis.

The quality or usefulness of PPG data from devices operating at relatively low sampling rates (or even those operating at higher rates) may be improved by performing local upsampling on the data. For example, quadratic interpolation may be performed the PPG data from the optical sensor 220 to generate a curve to fit each PPG heart beat pulse profile. Such interpolated curve data may be used to better approximate the locations of maxima, minima, or other values within the curve. In one example, a sampling rate of 100 Hz is combined with ongoing 3-point quadratic interpolation of the incoming PPG data to provide an enhanced PPG data curve without requiring a relatively high sampling rate. Other alternatives will be apparent to persons of ordinary skill in the art in view of the present disclosure.

Estimations of vital signs that are evaluated by the device 100 may be indicated to the wearer on the display 116. For example, the display 116 may comprise a multifunctional LED screen having different modes of operation to display different vital sign data. Mode selection may be performed using any suitable input, such as a dedicated mode button or a multifunction button. One or more of the wearer's heart rate, respiration rate, blood oxygen level, or other vital signs may be indicated on the display 116 at any given time. In one example, heart rate and respiration rate may be numerically indicated on the display 116. A version of the PPG data also may be displayed on the screen in the form of a pulse curve.

The sensor system 210 also preferably includes a motion sensor 222, such as a multi-axis accelerometer, to monitor the physical movement of the device 100, and thus the wearer. The motion sensor 222 may comprise, for example, an intelligent, low-power, 3/6/9-axis accelerometer with 12 bits of resolution. The resolution of the motion sensor 222 (i.e., the range, sensitivity and sampling rate of acceleration readings) may be selected as desired. The MIS2DH MEMS digital output motion sensor available from STMicroelectronics of Geneva, Switzerland is one example of an accelerometer that may be used in embodiments, but other devices may be used.

Various additional sensors may be provided to the sensor system 210. For example, a Global Positioning System (GPS) unit may be integrated into the device 100 to evaluate the location of the device 100. Such as GPS device may operate continuously, or may be activated at certain times, such as when an emergency alert is activated. A temperature sensor also may be provided to detect the wearer's temperature or an environmental temperature. As another example, a proximity sensor maybe provided to detect whether an object is adjacent the inner surface 108 of the housing 102. A galvanic skin response sensor also may be provided, if desired. Other alternatives will be apparent to persons of ordinary skill in the art in view of the present disclosure.

The communication system 212 may include one or more of a wireless communication interface 224 and a wired communication interface 226. The wireless communication interface 224 may include a transceiver (e.g. an integrated transmitting and receiving device or a paired arrangement of a transmitter and a separate receiver), or it may include only a transmitter. The wireless communication interface 224 preferably is operable to communicate directly with one or more emergency service providers. For example, the wireless communication interface 224 may comprise a digital transceiver operating under the Global System for Mobile Communications (GSM) protocol to communicate directly between the device 100 and a digital cellular network. The device 100 also may include a Subscriber Identity Module (SIM) card slot to receive user credentials or subscription information. The SIM card slot may be user-accessible, but where the device 100 is used in institutional settings (e.g., as a fall monitor in a hospital), the SIM card slot may be sealed to prevent ready access. The wireless communication interface 224 also may comprise any number of other communications devices using various different communication protocols. Examples include, but are not limited to: Bluetooth wireless transceivers, Wi-Fi 802.11 transceivers, Near Field Communication (NFC) transceivers, Zigbee transceivers, and radio frequency (RF) transceivers operating in any suitable frequency range, so on.

The wireless communication interface 224 may communicate directly with an existing global communication network. For example, GSM modules can establish communications with existing cellular networks, and Wi-Fl communication modules can establish voice-over Internet protocol (VoIP) communications, in respective manners that are known in the art. This may be desirable in applications where the device 100 is intended to be worn at a variety of different locations. In other examples, it may be necessary to provide an intermediary communication device to communicate with an existing global communication network. For example, the wearable biosensor device 100 may have a Bluetooth or NFC communication module that communicates with a cellular telephone or a local network to gain access to a global network. In other examples, the wireless communication interface 224 may be configured to connect only to a particular communication network. For example, the device 100 may have a Wi-Fi communication module that is configured to communication with a hospital network in which the device 100 is used. As another example, the device 100 may have a GSM module that is configured to communicate only with a particular network of call centers or medical response facilities. Combinations of these examples may be used, and other variations will be apparent to persons of ordinary skill in the art in view of this disclosure.

The wired communication interface 226 may include one or more connectors to interface the device 100 with an external processor or communication device. For example, a mini-USB or other port may be provided for establishing a wired communication link with a local computer. The wired communication interface 226 also may be used to establish a wired connection to an external portable communication device, such as a smartphone or the like that is carried on the user's person. When connected in this manner, the external portable communication device may be used to send emergency alerts to emergency service providers, and it may not be necessary for the device 100 to have a wireless communication interface 224 or the wireless communication device 224 may be temporarily disabled to conserve battery power.

The wireless communication interface 224 and the wired communication interface 226 may be used for various purposes in addition to sending emergency alerts. For example, one or both of the communication interfaces 224, 226 may be used to send configuration settings to the device 100, to provide software or firmware updates, to transmit data logs, and so on.

The selection of specific devices, electrical connections, drivers and control algorithms for the user interface system 208, sensor system 210 and communication system 212 will be understood by persons of ordinary skill in the art, and need not be described herein. Examples may include all or only some of the devices described above, and other alternatives and configurations will be apparent to persons of ordinary skill in the art in view of the present disclosure.

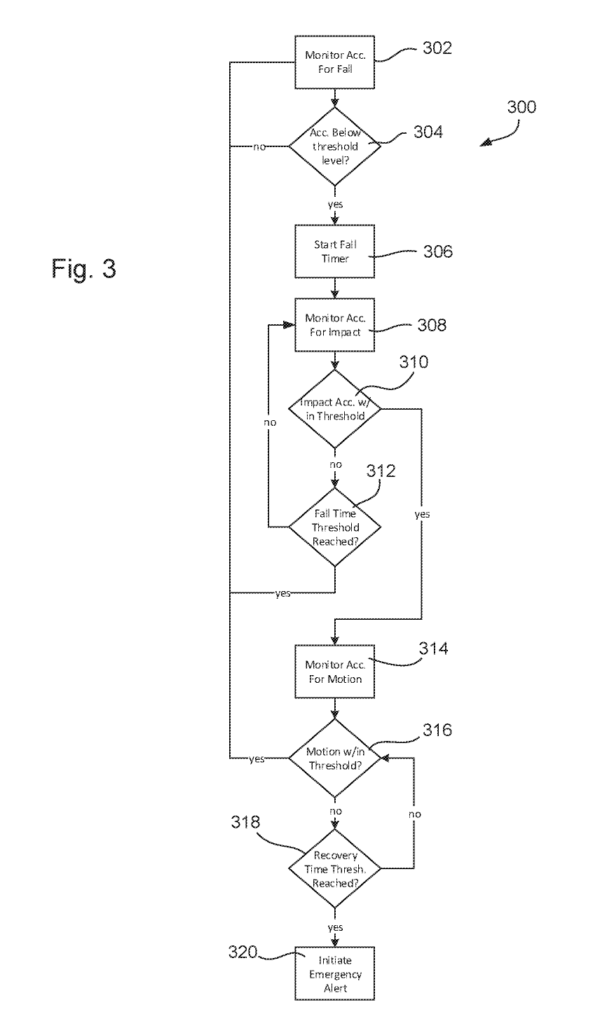

The wearable biosensor device 100 preferably is configured as a fall detector and emergency alert device. A significant problem in a fall detection system is the ability to differentiate between events that might require medical assistance and events that do not. A high incidence of false positives can reduce the utility of a device, lead to user dissatisfaction, and generate unnecessary medical service costs. FIG. 3 illustrates an example of a fall detection process 300 that may be performed by the processor 202 to detect falls and differentiate between threatening and non-threatening situations before generating an emergency alert.

The fall detection process 300 begins in step 302, in which the device 100 monitors a motion sensor 222 to determine whether a fall has occurred. As noted above, the motion sensor 222 may comprise a multi-axis accelerometer, a gyroscope, or other any other suitable device. The motion sensor 222 may be monitored continuously (i.e., at the maximum polling rate available to the system as limited by the processor 202 or other relevant components of the system), or at another predefined rate (e.g., 100 Hz). Monitoring also may be performed passively by effectively ignoring (i.e., not actively polling) the motion sensor 222 until the motion sensor 222 provides an interrupt signal indicating that an acceleration value above or below a certain threshold has been detected. For example, the motion sensor 222 may include an internal wake-up circuit that renders the motion sensor inactive until it experiences a minimum variation in acceleration values in one or more directions.

Acceleration data from the motion sensor 222 is evaluated to determine whether it includes indicia of a fall. In particular, it has been determined that persons experiencing falls typically register a reduction in acceleration as compared to the normal acceleration imposed by gravitational pull (i.e., 32.2 feet/second² or 9.81 meters/second²), which is referenced herein as “g”. A typical fall can be detected by a sudden transition from a steady gravitational acceleration value to a reduced acceleration value caused when the device 100 starts moving towards the ground. Thus, in step 304 the processor 202 evaluates the motion sensor data to determine whether the acceleration magnitude reduces to a predetermined value below gravitational acceleration. In some examples, this predetermined value may be an acceleration of 0.5 g or less, or 0.25 g or less, or 0 g. It will be appreciated that other examples may use different values, and the selection of the threshold value may be determined using empirical studies or other techniques. The threshold value may vary from example to example due to different preferences in sensitivity of the device 100 and tolerance for false positive readings. If the value is above the predetermined value, the processor 202 returns to monitoring the motion sensor 222 for a fall. If the value is below the predetermined value, the process moves to step 306.

Steps 306 through 312 define a process for filtering out false positive fall detections by determining whether the fall potentially detected in step 304 is followed by a significant impact within a predetermined amount of time. In step 306, the processor 202 begins a fall timer. The fall timer may comprise any conventional clock process to begin counting time immediately after the acceleration data falls below the threshold value. In step 308 (which may begin at any time after step 304), the processor 202 monitors the motion sensor data.

In step 310, the motion sensor data is assessed to determine whether the device 100 experiences an acceleration indicative of an impact, such as striking a floor or other object. Step 310 may be performed by comparing acceleration data from the motion sensor 222 to a threshold acceleration magnitude selected to represent a likely post-fall impact event. This acceleration magnitude may be, for example, 1.5 g, 2.0 g or 3.0 g of combined acceleration. The magnitude of this threshold value may be determined empirically. For example, data representing injury-causing falls may be evaluated to determine typical acceleration values. The threshold value also may be adjusted according to preferences such as a desire to include more or less of the likely statistical distribution of injury-causing impacts into the threshold value. The comparison performed in step 310 may be done on a per-axis basis or on a combined acceleration basis. For example, the comparison may be between the total combined acceleration magnitude in all axes and a total threshold value, or it may be a comparison between an acceleration value in the gravitational direction and a corresponding threshold value. Other alternatives will be apparent to persons of ordinary skill in the art in view of the present disclosure.

If no acceleration above the threshold is detected in step 310 during a particular processing cycle, the process moves to step 312, in which the fall timer is checked to determine whether a predetermined amount of time has passed since the possible fall was detected in step 304. The use of a threshold time in step 312 is expected to be helpful to filter out situations in which a wearer might seem to fall, but does not experience an impact in the time expected for the fall to complete. For example, a wearer might move in a way that only appears to be a fall (e.g., rapidly waving the arm to which the device 100 is attached), or the wearer might fall but recover from the fall without experiencing a detrimental impact. The threshold time may be, for example, 0.25 seconds, 0.5 seconds, 0.75 seconds or 1.0 seconds. The use of the timer also prevents a later innocuous impact (e.g., bumping the device 100 against a table long after a possible fall is detected in step 304) from being considered a post-fall impact requiring medical attention. As with the threshold acceleration used in steps 304 and 310, empirical testing or other factors may be used to arrive at different threshold time values. If the threshold time is not reached in step 312, the process returns to step 308 and the fall timer is incremented. If the threshold time is reached in step 312, then the process continues to step 302.

If an acceleration above the threshold value is detected in step 310 before the fall timer runs out, the process moves to step 314. In this step, the processor 202 monitors the motion sensor 222 to determine whether the wearer is moving after the presumably detected fall and impact. Such motion can indicate fall recovery or the resumption of normal movement. Such motion can also indicate that the supposed fall and impact were actually not a fall and/or an impact. Regular movement after a fall also indicates that the wearer is able to continue operating normally and is able to seek assistance without help.

In step 316, the acceleration data from the motion sensor 222 is again compared to a predetermined acceleration threshold. In this instance, it is presumed that the wearer has come to a rest, such that the normal g value of acceleration in the global vertical direction is registered by the sensors. Thus, motion of the wearer will be evaluated as variations from the normal g value. As with the other values discussed above, the post-impact motion threshold value may be determined empirically or by other means. In some examples, the threshold value may be a deviation from g of more than 0.05 g, more than 0.10 g, or more than 0.25 g. If movement indicative of a fall recovery (or the absence of a fall or impact in the first place) is detected in step 316, the process returns to step 302.

If no movement or insufficient movement is detected in step 316, the process proceeds to step 318, in which the time value of the fall timer (or a separate timer activated at some other point in the process) is compared to a threshold recovery time value. Again, the threshold recovery time may be established empirically or by other methods. For example, it may be a five second timer, a ten second timer or a fifteen second timer. Process steps 316 and 318 continue to loop until motion indicating recovery is detected, or the recovery timer runs out. If the recovery timer elapses without recovery movement being detected, the process moves to step 320 to initiate an emergency alert.

As noted above, the various threshold values used in the steps of the fall detection process 300 may be established or modified as desired. The threshold value comparisons may comprise simple one-to-one value comparisons, or they may comprise more detailed algorithms. For example, in step 316, the evaluation of post-impact motion may involve a comparison of acceleration data from the motion sensor 222 to motion data immediately prior to the detection of a fall in step 304 to determine whether the wearer has apparently resumed the activities performed before the fall (e.g., accelerations indicative of a hand swinging during walking or running activity). As another example, step 316 may compare an average value of accelerations over time to determine whether the wearer is continuing to move in a regular manner, suggesting good health. Thus, the comparison may include additional mathematical or statistical evaluations beyond simple one-to-one magnitude comparisons.

One or more of the threshold values also may be developed via learning routines. For example, the device 100 may be configured to operate in a learning mode in which it observes the wearer's movements to generate a movement profiles characteristic of the wearer or the wearer's particular activities. Those values can be used to weight or replace the baseline threshold values provided in the device. The threshold values also may be modified manually to account for different operating conditions. For example, a “sports” mode may be activated to suppress the likelihood of activating an emergency alert during strenuous physical exercise by increasing or modifying one or more of the threshold values.

As another example, in some cases it may not be desirable to filter the data to prevent false positives as strictly or at all. One such case is where the wearer is a medical patient is confined to a bed or unconscious, and there is little likelihood that a fall or an impact can be falsely detected. In such cases, the threshold in step 304 may be lower. Another case is where the wearer is a person particularly sensitive to injury from falls, and is more likely to be injured even by relatively minor impacts. In this case, the threshold value in step 310 may be decreased. The device 100 may be pre-programmed with these and other modes to account for various different situations in which the device might be used.

Other examples may include additional data to assess the fall condition or post-fall conditions. For example, steps 314 and 316 may be replaced or supplemented by process steps for monitoring the wearer's heart rate, respiration rate, or other vital signs, to check for indicia of trauma.

The device 100 also may initiate an audible query to the wearer upon detecting what is believed to be a fall and impact situation. For example, upon reaching step 314, the processor 202 may activate a speaker on the device 100 and transmit an audio message stating that a fall has been detected and prompting the wearer to confirm or dismiss the initiation of an emergency alert. The wearer may then reply, if able, by speaking a confirmation or dismissal message into a microphone on the device 100, or entering instructions via a tactile input such as a button or touchscreen. Other alternatives will be apparent to persons of ordinary skill in the art in view of the present disclosure.

It is also envisioned that the device may operate as a manually-operated emergency alert device, or as an alert device that calls medical services upon determining certain physiological conditions of the wearer (e.g., a pattern or irregular pulse variations indicating a serious medical condition).

When an alert situation is detected, a communications system within the system can be activated to transmit data to emergency service providers. Such data may include pre-determined messages (e.g., an alert to go to a particular address, or to go to the last known address of the device), information about the wearer's physiological condition (e.g., streaming PPG data or the like), and so on.

Embodiments may be used in a number of ways. For example, a biosensor as disclosed herein can be used to monitor heart rate, respiration rate, oxygen saturation, blood pressure, body temperature, skin galvanic conditions, electrical impulses reflective of pulse rate or muscular contraction, body movement, and so on. The sensors may be operated continuously or intermittently (e.g., on demand or at predetermined intervals). The device also may be remotely operated to perform remote data collection and analysis. For example, a patient undergoing medical care can be monitored remotely by a doctor that initiates remote data collection and review wirelessly though the Internet or cellular networks.

The present disclosure describes a number of new, useful and nonobvious features and/or combinations of features that may be used alone or together. The embodiments described herein are all exemplary, and are not intended to limit the scope of the inventions. It will be appreciated that the features shown and described in documents incorporated herein by reference may be added to embodiments in a manner corresponding to the use of such features in the incorporated references. It will also be appreciated that the inventions described herein can be modified and adapted in various ways, and all such modifications and adaptations are intended to be included in the scope of this disclosure and the appended claims.

1. A fall detecting device comprising:
a housing configured to be positioned adjacent a wearer's body;
a processor;
a sensor system operatively connected to the processor and configured to detect a physiological condition of the wearer's body;
a motion sensor operatively connected to the processor;
a wireless communication module operatively connected to the processor;
a memory operatively connected to the processor, the memory storing computer readable instructions that, when executed, cause the processor to:
monitor the motion sensor to detect a decrease in acceleration in the gravitational direction below a first predetermined threshold;
start a timer upon detecting the decrease in acceleration;
monitor the motion sensor to determine whether the motion sensor signals a fall-indicative acceleration above a second predetermined level prior to the timer reaching a first predetermined time;
monitor the motion sensor to determine whether the motion sensor signals a recovery-indicative acceleration above a third predetermined level prior to the timer reaching a second predetermined time; and
activate the wireless communication module to initiate an emergency alert if the fall-indicative acceleration is detected and the recovery-indicative acceleration is not detected.

2. The fall detecting device of claim 1, wherein the recovery-indicative acceleration comprises an average value of accelerations over time.

3. The fall detecting device of claim 1, wherein the computer readable instructions further cause the processor to activate a speaker to prompt the wearer to confirm or dismiss an initiation of an emergency alert.

4. The fall detecting device of claim 1, wherein the sensor system comprises a photoplethysmographic sensor.

5. The fall detecting device of claim 4, wherein the computer readable instructions further cause the processor to:
operate the photoplethysmographic sensor to detect blood flow information;
determine a heart rate of the wearer based on the blood flow information; and
display the heart rate on a user interface.

6. The fall detecting device of claim 4, wherein the computer readable instructions further cause the processor to:
operate the photoplethysmographic sensor to detect a first set of blood flow information for a first detection time period following an initial activation of the fall detecting device;
determine a first heart rate of the wearer during the first detection time period based on the first set of blood flow information;
display the first heart rate on a user interface;
operate the photoplethysmographic sensor to detect a second set of blood flow information for a second detection time period following the first detection time period;
determine a second heart rate of the wearer during the second detection time period based on the second set of blood flow information; and
display the second heart rate on the user interface.

7. The fall detecting device of claim 6, wherein the second period of time is longer than the first period of time.

8. The fall detecting device of claim , further comprising a user interface on the housing.

9. The fall detecting device of claim 8, wherein the user interface includes a manually-operable emergency alert input operable to cause the processor to activate the wireless communication module to initiate an emergency alert.

10. The fall detecting device of claim 8, wherein the user interface is configured to provide visual information regarding the physiological condition of the wearer's body.

11. The fall detecting device of claim 1, further comprising a battery operatively connected to the processor and configured to power the fall detecting device.

12. The fall detecting device of claim 1, wherein the motion sensor comprises a multi-axis accelerometer.

13. A fall detection method for a fall detecting device having at least one motion sensor, the method comprising:
monitoring a physiological condition of a wearer;
monitoring the motion sensor to detect a decrease in acceleration in the gravitational direction below a first predetermined threshold;
starting a timer upon detecting the decrease in acceleration;
monitoring the motion sensor to determine whether the motion sensor signals a fall-indicative acceleration above a second predetermined level prior to the timer reaching a first predetermined time;
monitoring the motion sensor to determine whether the motion sensor signals a recovery-indicative acceleration above a third predetermined level prior to the timer reaching a second predetermined time; and
activating a wireless communication module to initiate an emergency alert if the fall-indicative acceleration is detected and the recovery-indicative acceleration is not detected.

14. The fall detecting method of claim 12, wherein the recovery-indicative acceleration comprises an average value of accelerations over time.

15. The fall detecting method of claim 12, further comprising activating a speaker to request confirmation or dismissal of an initiation of an emergency alert.
